(19) United States
(12) Patent Application Publication
(10) Pub. No.: US 2022/0387057 A1
(43) Pub. Date: Dec. 8, 2022

(54) Hub and Valve Systems for an Aspiration Catheter

(71) Applicant: Retriever Medical, Inc., Las Vegas, NV (US)

(72) Inventors: Michael Bruce Horowitz, Naples, FL (US); Benjamin William Bobo, Las Vegas, NV (US); Brandon Matthew Repko, Mars, PA (US); Hieu Le, Fountain Valley, CA (US)

(21) Appl. No.: 17/809,517

(22) Filed: Jun. 28, 2022

Related U.S. Application Data
(63) Continuation-in-part of application No. 17/572,138, filed on Jan. 10, 2022, now Pat. No. 11,382,643.
Application No. 17/572,138 is a continuation of application No. 17/450,977, filed on Oct. 14, 2021.
Application No. 17/450,977 is a continuation-in-part of application No. 17/572,206, filed on Jan. 10, 2022.
Application No. 17/572,206 is a continuation of application No. 17/450,978, filed on Oct. 14, 2021.
(60) Provisional application No. 63/215,579, filed on Jun. 28, 2021.
(60) Provisional application No. 63/364,168, filed on May 4, 2022.
(60) Provisional application No. 63/268,094, filed on Feb. 16, 2022.
(60) Provisional application No. 63/260,406, filed on Aug. 19, 2021.
(60) Provisional application No. 63/215,724, filed on Jun. 28, 2021.
(60) Provisional application No. 63/215,579, filed on Jun. 28, 2021.
(60) Provisional application No. 63/215,573, filed on Jun. 28, 2021.
(60) Provisional application No. 63/215,587, filed on Jun. 28, 2021.
(60) Provisional application No. 63/215,583, filed on Jun. 28, 2021.
(60) Provisional application No. 63/215,565, filed on Jun. 28, 2021.
(60) Provisional application No. 63/092,428, filed on Oct. 15, 2020.
(60) Provisional application No. 63/260,406, filed on Aug. 19, 2021.
(60) Provisional application No. 63/215,724, filed on Jun. 28, 2021.
(60) Provisional application No. 63/215,579, filed on Jun. 28, 2021.
(60) Provisional application No. 63/215,573, filed on Jun. 28, 2021.
(60) Provisional application No. 63/215,587, filed on Jun. 28, 2021.
(60) Provisional application No. 63/215,583, filed on Jun. 28, 2021.
(60) Provisional application No. 63/215,565, filed on Jun. 28, 2021.
(60) Provisional application No. 63/092,428, filed on Oct. 15, 2020.

(51) Int. Cl. A61B 17/22; A61B 17/221

(52) U.S. Cl. CPC A61B 17/22032; A61B 17/221; A61B 2017/00867

(57) ABSTRACT

A valve system for a catheter, such as an aspiration catheter, is disclosed and includes a valve having a proximal end and a distal end. The distal end of the valve is coupled to the catheter. The valve has a flexible lumen in a first state that is constricted or twisted. The valve system is housed within a hub, where actuation of a button on the hub causes the proximal and distal ends of the valve to rotate relative to each other to put the lumen in a second state that is unconstricted or untwisted state.

### CROSS-REFERENCE

The present application relies on, for priority, U.S. Patent Provisional Application No. 63/215,579, titled “Hub and Valve Systems for An Aspiration Catheter” and filed on Jun. 28, 2021, and is herein incorporated by reference in its entirety.

The present application also relies on, for priority, U.S. Patent Provisional Application No. 63/364,168, titled “Clot Removal Methods and Devices with Specialized Clot Removal Elements” and filed on May 4, 2022, and U.S. Patent Provisional Application No. 63/268,094, titled “Methods and Devices for Removing and Filtering Clots to Isolate Blood for Reinfusion into a Patient” and filed on Feb. 16, 2022.

The present application is also a continuation-in-part application of U.S. patent application Ser. No. 17/572,138, titled “Clot Removal Methods and Devices with Multiple Independently Controllable Elements” and filed on Jan. 10, 2022, which is a continuation application of U.S. patent application Ser. No. 17/450,977, of the same title and filed on Oct. 14, 2021.

The present application is also a continuation-in-part application of U.S. patent application Ser. No. 17/572,206, titled “Clot Removal Methods and Devices with Multiple Independently Controllable Elements” and filed on Jan. 10, 2022, which is a continuation application of U.S. patent application Ser. No. 17/450,978, of the same title and filed on Oct. 14, 2021.

Both U.S. patent application Ser. No. 17/450,977 and U.S. patent application Ser. No. 17/450,978 rely on, for priority, the following provisional applications:

U.S. Patent Provisional Application No. 63/260,406, titled “Catheter Based Retrieval Device” and filed on Aug. 19, 2021;

U.S. Patent Provisional Application No. 63/215,724, titled “Device and Method of Using the Device for Repairing A Pathological Connection Between Two Anatomical Structures” and filed on Jun. 28, 2021;

U.S. Patent Provisional Application No. 63/215,579, titled “Hub and Valve Systems for an Aspiration Catheter” and filed on Jun. 28, 2021;

U.S. Patent Provisional Application No. 63/215,573, titled “Aspiration Catheters and Methods of Use Thereof” and filed on Jun. 28, 2021;

U.S. Patent Provisional Application No. 63/215,587, titled “Vascular Closure Devices and Methods of Using Thereof” and filed on Jun. 28, 2021;

U.S. Patent Provisional Application No. 63/215,583, titled “Catheters with Expandable and Collapsible Lumens” and filed on Jun. 28, 2021;

U.S. Patent Provisional Application No. 63/215,565, titled “Catheter Based Retrieval Device” and filed on Jun. 28, 2021; and

U.S. Patent Provisional Application No. 63/092,428, titled “Catheter Based Retrieval Device with Proximal Body Having Axial Freedom of Movement” and filed on Oct. 15, 2020.

All of the above-mentioned patents and applications are hereby incorporated by reference in their entirety.

### FIELD

The disclosure generally relates to hub and valve mechanisms that enable the application and removal of suction to an aspiration catheter through the valve.

### BACKGROUND

Many medical procedures, such as, for example, mechanical thrombectomy, involve introduction of at least one medical instrument into arterial, venous and neural systems so that the medical instrument may be advanced to a body location requiring diagnosis or treatment. For example, a guide catheter may be advanced through a patient's vasculature (such as those in the brain) to a desired treatment location and the medical instrument advanced through the guide catheter for the removal of occlusions, such as thrombi. The guide catheter is often inserted through an aspiration catheter in order to purge occlusion material removed by the medical instrument from the treatment location.

A hub is typically coupled to a proximal end of the aspiration catheter via a valve. The hub is operated to control opening and closing of the valve thereby manipulating application of suction through the aspiration catheter.

There exists a continuing need for improved valve and hub systems that are easy to operate. There is also a need for hubs that allow for single-handed manipulation to open and seal associated valves when desired.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a valve system configured to be coupled to a catheter, the valve system comprising: a valve having a proximal end and a distal end, wherein the distal end of the valve is coupled to a lumen of the catheter, wherein the valve comprises a flexible material extending between the proximal end and distal end and wherein the valve lumen has a first twisted state and a second untwisted state; and a hub having a body, a rack, a pinion physically coupled to the rack, and an actuator, wherein the body encloses the rack, wherein the pinion is coupled to the proximal end of the valve, and wherein the actuator is coupled to the rack such that applying a force to the actuator moves the rack which causes the pinion to rotate in a first direction and thereby causes the lumen to untwist and adopt the second untwisted state.

Optionally, the actuator is coupled to the rack such that removing a pressure or physical force from the actuator automatically causes the rack to move and thereby rotate the pinion in a second direction to cause the lumen to adopt the first twisted state.

Optionally, the flexible material comprises at least one of polytetrafluoroethylene, urethane or silicone.

Optionally, the valve further comprises at least one gear in the hub, wherein the pinion is coupled to the proximal end of the valve through said at least one gear.

Optionally, the valve is reinforced with one or more members comprising at least one of stainless steel, nitinol or nylon.

Optionally, the valve lumen has an inner diameter ranging from 0.01 inches to 2 inches. Optionally, the valve lumen has a wall thickness ranging from 0.0002 inches to 0.125 inches. Optionally, the valve lumen has a length ranging from 0.01 inches to 2 inches.

Optionally, the valve system further comprises at least one gear in the hub, wherein the rack is coupled to the pinion through said at least one gear.

Optionally, the valve lumen is configured to be rotated by an angle ranging from 90 degrees to 360 degrees in order to place the lumen in the first state.

The present specification also discloses a valve system configured to be coupled to a catheter, the valve system comprising: a valve having a proximal end and a distal end, wherein the distal end of the valve is coupled to a lumen of the catheter, wherein the valve comprises a flexible material defining the proximal end, distal end and lumen extending therebetween and wherein the valve lumen has a first twisted state and a second untwisted state; and a hub having a body, a first rack, a second rack, a pinion configured to concurrently engage with the first rack and the second rack, and an actuator, wherein the second rack is coupled to the proximal end of the valve, and wherein the actuator is coupled to the pinion such that applying a force to the actuator rotates the pinion, moves the pinion along the first rack, causes the second rack to move in a first direction, thereby causing the valve lumen to untwist and adopt the second untwisted state.

Optionally, the first rack is configured to be stationary and the second rack is configured to be movable.

Optionally, the first twisted state forms a seal with respect to the catheter.

Optionally, the second untwisted state allows for a flow of fluid or a passage of a device into the catheter.

Optionally, the flexible material comprises at least one of PTFE, ePTFE, urethane or silicone. Optionally, the hub further comprises at least one of stainless steel members, nitinol members or Nylon members.

Optionally, the valve lumen has an inner diameter ranging from 0.01 inches to 2 inches. Optionally, the valve lumen has a wall thickness ranging from 0.0002 inches to 0.125 inches. Optionally, the valve lumen has a length ranging from 0.01 inches to 2 inches.

Optionally, the actuator is coupled to the pinion such that removing a force from the actuator rotates the pinion, moves the pinion along the first rack, and causes the second rack to move in a second direction opposite the first direction, thereby causing the valve lumen to twist and adopt the first twisted state. Optionally, the flexible lumen is configured to be rotated by an angle ranging from 90 degrees to 360 degrees in order to constrict the lumen in the first twisted state.

The present specification also discloses a valve system for an aspiration catheter, the system comprising: a valve having a proximal end and a distal end, wherein the distal end of the valve is coupled to the aspiration catheter, and wherein the valve is a tube having a flexible lumen, said lumen being in a first state; a hub having a body enclosing a cylinder and a slider positioned on the body, said cylinder having a substantially helical groove formed on an outer surface of the cylinder, said slider being constrained to move within a slot formed in the body and said slider having a pin that engages with and tracks the groove; wherein moving the slider distally causes the pin to move within the groove and rotate the cylinder in a first direction, wherein the rotation of the cylinder in the first direction causes the proximal and distal ends of the valve to rotate relative to each other, and wherein said relative rotation puts said lumen in a second state.

Optionally, the first state corresponds to said lumen being constricted. Optionally, the second state corresponds to said lumen being open.

Optionally, the tube is made of at least one of PTFE, ePTFE, urethane or silicone. Optionally, the tube is reinforced with stainless steel, nitinol and/or Nylon members.

Optionally, the tube has an inner diameter ranging from 0.01 inches to 2 inches. Optionally, the tube has a wall thickness ranging from 0.0002 inches to 0.125 inches. Optionally, the tube has a length ranging from 0.01 inches to 2 inches.

Optionally, moving the slider proximally causes the pin to move within the groove and rotate the cylinder in a second direction opposite to the first direction, wherein the rotation of the cylinder in the second direction causes the proximal and distal ends of the valve to rotate relative to each other, and wherein said relative rotation puts said lumen in a second state. Optionally, the tube is configured to be rotated by an angle ranging from 90 degrees to 360 degrees in order to constrict the lumen in the first state.

The present specification also discloses a syringe configured to automatically hold a plunger in a drawn back state within a barrel until released, the syringe comprising: a latch base attached to an outer surface of the barrel; and a lever movably coupled to the latch base, wherein the lever is pivoted relative to the latch base, said lever having an arm at one end and a hammer at another end, wherein a distal end of the plunger includes a flange, and wherein moving the plunger proximally causes the hammer to fall into place behind the flange thereby holding the plunger in the drawn back state.

Optionally, depressing the arm of the lever causes the hammer to be pulled up thereby releasing the plunger to be pushed forward.

The present specification also discloses a valve system for an aspiration catheter, the system comprising: a valve having a proximal end and a distal end, wherein the distal end of the valve is fixedly coupled to the aspiration catheter, wherein the valve comprises a tube having a flexible lumen that extends between the proximal end and the distal end, and wherein the lumen is in a first constricted state; and a hub having a body comprising: a rack; a pinion engaged with the rack; a stepped-up gear engaged with the pinion; and an actuator positioned on the body, wherein the rack is coupled to the actuator and the stepped-up gear is coupled to the proximal end of the valve; and wherein the actuator, rack, pinion, and stepped-up gear are configured such that applying a force to the actuator displaces the rack along a linear axis which rotates the pinion in a first direction and moves the pinion along the rack, which, in turn, causes the stepped-up gear to rotate in the first direction, which, in turn, causes the proximal end to rotate relative to the distal end, and wherein said relative rotation puts said flexible lumen in a second open state.

Optionally, the first constricted state creates a seal around the aspiration catheter.

Optionally, the second open state allows flow of fluid or passage of devices to the aspiration catheter.

Optionally, a material of the tube comprises at least one of polytetrafluoroethylene, urethane or silicone. Optionally, the tube further comprises at least one of stainless steel members, nitinol members or Nylon members.

Optionally, the flexible lumen has an inner diameter ranging from 0.01 inches to 2 inches.

Optionally, the flexible lumen has a wall thickness ranging from 0.0002 inches to 0.125 inch.

Optionally, the flexible lumen has a length ranging from 0.01 inches to 2 inches.

Optionally, the actuator is coupled to the rack such that removing a pressure or physical force from the actuator automatically moves the pinion along the rack causing the stepped-up gear to rotate in a second direction opposite to the first direction, wherein the rotation of the stepped-up gear in the second direction causes the proximal end of the valve to rotate relative to the distal end, and wherein said relative rotation puts said flexible lumen back in the first constricted state. Optionally, the flexible lumen is configured to be rotated by an angle ranging from 90 degrees to 360 degrees in order to constrict the lumen in the first state.

Optionally, the present specification also disclose a method of selectively applying suction to a catheter, comprising: providing a catheter and a valve system coupled to the catheter, wherein the valve system comprises: a valve having a proximal end and a distal end, wherein the distal end of the valve is coupled to a lumen of the catheter, wherein the valve comprises a flexible material defining the proximal end, distal end and lumen extending therebetween and wherein the valve lumen has a first twisted state and a second untwisted state; and a hub having a body, a rack, a pinion physically engaging said rack, and an actuator, wherein the body encloses the rack, wherein the pinion is coupled to the proximal end of the valve, and wherein the actuator is coupled to the rack such that applying a force to the actuator moves the rack and rotates the pinion in a first direction causing the valve lumen to untwist and adopt the second untwisted state; and applying force to said actuator to cause the valve lumen to untwist and adopt the second untwisted state; using a syringe coupled to the catheter, applying suction to the catheter; and removing the force from said actuator to cause the valve lumen to twist and adopt the first twisted state.

Optionally, the actuator is coupled to the rack such that the removal of the force from the actuator moves the rack and rotates the pinion in a second direction causing the valve lumen to twist and adopt the first twisted state.

The present specification also discloses a method selectively applying suction to a catheter, comprising: providing a catheter and a valve system coupled to the catheter, wherein the valve system comprises: a valve having a proximal end and a distal end, wherein the distal end of the valve is coupled to a lumen of the catheter, wherein the valve comprises a flexible material defining the proximal end, distal end and lumen extending therebetween and wherein the valve lumen has a first twisted state and a second untwisted state; and a hub having a body, a first rack, a second rack, a pinion configured to concurrently engage with the first rack and the second rack, and an actuator, wherein the second rack is coupled to the proximal end of the valve, and wherein the actuator is coupled to the pinion such that applying a force to the actuator rotates the pinion, moves the pinion along the first rack, causes the second rack to move in a first direction, thereby causing the valve lumen to untwist and adopt the second untwisted state; and applying force to said actuator to cause the valve lumen to untwist and adopt the second untwisted state; using a syringe coupled to the catheter, applying suction to the catheter; and removing the force from said actuator to cause the valve lumen to twist and adopt the first twisted state.

Optionally, the actuator is coupled to the pinion such that the removal of the force from the actuator rotates the pinion, moves the pinion along the first rack, causes the second rack to move in a second direction opposite the first direction, thereby causing the valve lumen to twist and adopt the first twisted state.

The present specification also discloses a method of selectively applying suction to a catheter, comprising: providing a catheter and a valve system coupled to the catheter, wherein the valve system comprises: a valve having a proximal end and a distal end, wherein the distal end of the valve is coupled to the aspiration catheter, and wherein the valve comprises a tube having a flexible lumen, said lumen being in a first constricted state; and a hub having a body enclosing a cylinder and a slider positioned on the body, said cylinder having a substantially helical groove formed on an outer surface of the cylinder, said slider being constrained to move within a slot formed in the body and said slider having a pin that engages with and tracks the groove; wherein moving the slider distally causes the pin to move within the groove and rotate the cylinder in a first direction, wherein the rotation of the cylinder in the first direction causes the proximal and distal ends of the valve to rotate relative to each other, and wherein said relative rotation puts said lumen in a second unconstricted state; and applying force to said slider to cause the flexible lumen to unconstrict and adopt the second unconstrict state; using a syringe coupled to the catheter, applying suction to the catheter; and removing the force from said slider to cause the flexible lumen to twist and adopt the first constricted state.

The present specification also discloses a valve system for an aspiration catheter, the valve system comprising: a valve having a proximal end and a distal end, wherein the distal end of the valve is coupled to a lumen of the aspiration catheter, wherein the valve comprises a flexible material extending through a length of the valve and defining a lumen, and wherein the valve lumen is in a first state; and a hub having a body enclosing a rack engaged with an associated pinion, wherein the hub comprises a button positioned on the body, wherein the pinion is coupled to a proximal end of the valve and wherein the button is coupled to the rack; wherein the button is configured such that applying a force to the button moves the rack and rotates the pinion in a first direction causing the proximal and distal ends of the valve to rotate relative to each other, and wherein said relative rotation puts the valve lumen in a second state.

Optionally, the first state corresponds to said lumen being twisted such that the lumen is constricted.

Optionally, the second state corresponds to said lumen being untwisted such that the lumen is unconstricted.

Optionally, the flexible material comprises at least one of ePTFE, urethane or silicone. Optionally, the valve is reinforced with one or more members comprising at least one of stainless steel, nitinol or nylon.

Optionally, the valve lumen has an inner diameter ranging from 0.01 inch to 2 inches.

Optionally, the valve lumen has a wall thickness ranging from 0.0002 inch to 0.125 inch.

Optionally, the valve lumen has a length ranging from 0.01 inch to 2 inches.

Optionally, releasing of the button moves the rack and rotates the pinion in a second direction opposite to the first direction causing the proximal and distal ends of the valve to rotate relative to each other, wherein said relative rotation puts the lumen in the first state. Optionally, the tube is configured to be rotated by an angle ranging from 90 degrees to 360 degrees in order to constrict the lumen in the first state.

The present specification also discloses a valve system for an aspiration catheter, the system comprising: a valve having a proximal end and a distal end, wherein the distal end of the valve is coupled to the aspiration catheter, wherein the valve comprises a tube having a flexible lumen and wherein the lumen is in a first constricted state; and a hub having a body enclosing a first rack, a second rack and a pinion engaged with the first and second racks, wherein the hub comprises an actuator positioned on the body, wherein the pinion is coupled to the actuator and the second rack is coupled to the proximal end of the valve; wherein applying a force to the actuator rotates the pinion and moves the pinion along the first rack causing the second rack to move in a first direction, wherein the movement of the second rack in the first direction causes the proximal and distal ends of the valve to rotate relative to each other, and wherein said relative rotation puts said lumen in a second untwisted state.

Optionally, the first rack is stationary and the second rack is movable.

Optionally, the first constricted state creates a seal to the aspiration catheter.

Optionally, the second unconstricted state allows flow of fluid or passage of devices to the aspiration catheter.

Optionally, a material of the tube comprises at least one of ePTFE, urethane or silicone. Optionally, the tube further comprises at least one of stainless steel members, Nitinol members or Nylon members.

Optionally, the flexible lumen has an inner diameter ranging from 0.01 inch to 2 inches.

Optionally, flexible lumen has a wall thickness ranging from 0.0002 inch to 0.125 inch.

Optionally, the flexible lumen has a length ranging from 0.01 inch to 2 inches.

Optionally, removing force from the actuator rotates the pinion and moves the pinion along the first rack causing the second rack to move in a second direction opposite to the first direction, wherein said movement of the second rack in the second direction causes the proximal and distal ends of the valve to rotate relative to each other, and wherein said relative rotation puts said lumen back in the first constricted state. Optionally, the flexible lumen is configured to be rotated by an angle ranging from 90 degrees to 360 degrees in order to constrict the lumen in the first state.

The present specification also discloses a valve system for an aspiration catheter, the system comprising: a valve having a proximal end and a distal end, wherein the distal end of the valve is coupled to the aspiration catheter, and wherein the valve is a tube having a flexible lumen, said lumen being in a first state; a hub having a body enclosing a cylinder and a slider positioned on the body, said cylinder having a substantially helical groove formed on an outer surface of the cylinder, said slider being constrained to move within a slot formed in the body and said slider having a pin that engages with and tracks the groove; wherein moving the slider distally causes the pin to move within the groove and rotate the cylinder in a first direction, wherein the rotation of the cylinder in the first direction causes the proximal and distal ends of the valve to rotate relative to each other, and wherein said relative rotation puts said lumen in a second state.

Optionally, the first state corresponds to said lumen being constricted.

Optionally, the second state corresponds to said lumen being open.

Optionally, the tube is made of at least one of ePTFE, urethane or silicone. Optionally, the tube is reinforced with stainless steel, Nitinol or Nylon members.

Optionally, the tube has an inner diameter ranging from 0.01 inch to 2 inches.

Optionally, the tube has a wall thickness ranging from 0.0002 inch to 0.125 inch.

Optionally, the tube has a length ranging from 0.01 inch to 2 inches.

Optionally, moving the slider proximally causes the pin to move within the groove and rotate the cylinder in a second direction opposite to the first direction, wherein the rotation of the cylinder in the second direction causes the proximal and distal ends of the valve to rotate relative to each other, and wherein said relative rotation puts said lumen in a second state. Optionally, the tube is configured to be rotated by an angle ranging from 90 degrees to 360 degrees in order to constrict the lumen in the first state.

The present specification also discloses a syringe configured to automatically hold a plunger in a drawn back state within a barrel until released, the syringe comprising: a latch base attached to an outer surface of the barrel; and a lever movably coupled to the latch base, wherein the lever is pivoted relative to the latch base, said lever having an arm at one end and a hammer at another end, wherein a distal end of the plunger includes a flange, and wherein moving the plunger proximally causes the hammer to fall into place behind the flange thereby holding the plunger in the drawn back state.

Optionally, depressing the arm of the lever causes the hammer to be pulled up thereby releasing the plunger to be pushed forward.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A illustrates a retrieval device, in accordance with some embodiments of the present specification;

FIG. 1B illustrates a hub coupled to a catheter and a valve with a connected negative pressure source, in accordance with some embodiments of the present specification;

FIG. 1C illustrates another embodiment of a hub coupled to a catheter and a valve, connected to a negative pressure source, in accordance with the present specification;

FIG. 2 illustrates first and second cross-sectional views of a flexible lumen tube or valve, in accordance with some embodiments of the present specification;

FIG. 3 shows perspective, side and top views of a hub, including a single rack and pinion arrangement, for use with a flexible lumen tube or valve, in accordance with some embodiments of the present specification;

FIG. 4 shows a hub including a double rack and pinion arrangement, for use with a flexible lumen tube or valve, in accordance with some embodiments of the present specification;

FIG. 5 shows perspective, side and top views of a hub including a twist mechanism driven by a cammed slider, for use with a flexible lumen tube or valve, in accordance with some embodiments of the present specification;

FIG. 6 illustrates another embodiment of a hub included in a valve mechanism, in accordance with the present specification;

FIG. 7 illustrates yet another embodiment of a hub included in a valve mechanism, in accordance with the present specification;

FIG. 8 shows first, second and third views of a locking syringe, in accordance with some embodiments of the present specification; and

FIG. 9 is a flow chart illustrating an exemplary method for effectuating aspiration using an aspiration catheter connected to an aspiration port of a hub with a locking syringe, in accordance with some embodiments of the present specification.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

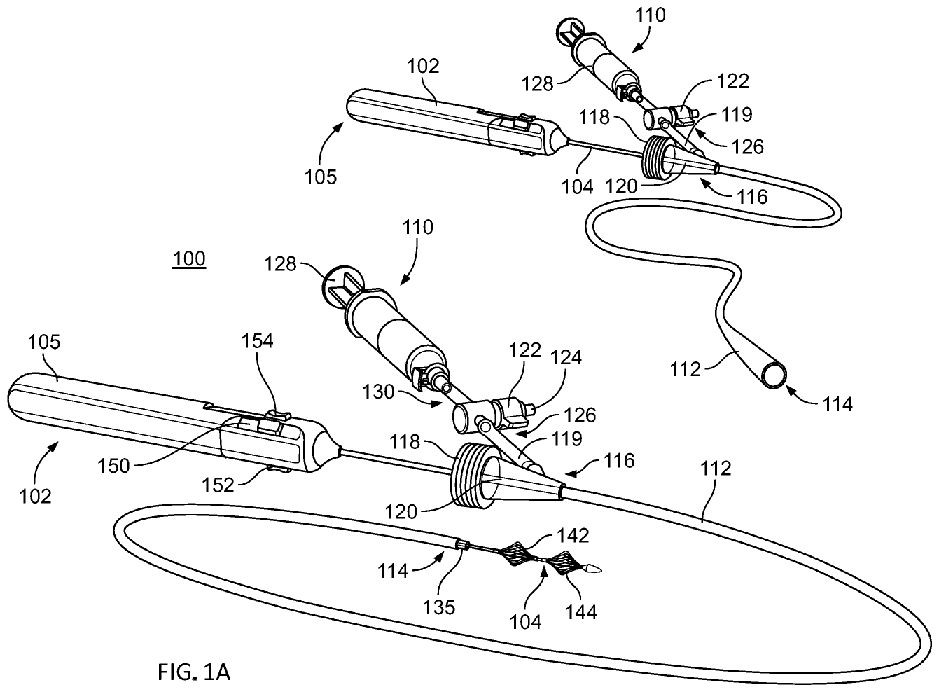

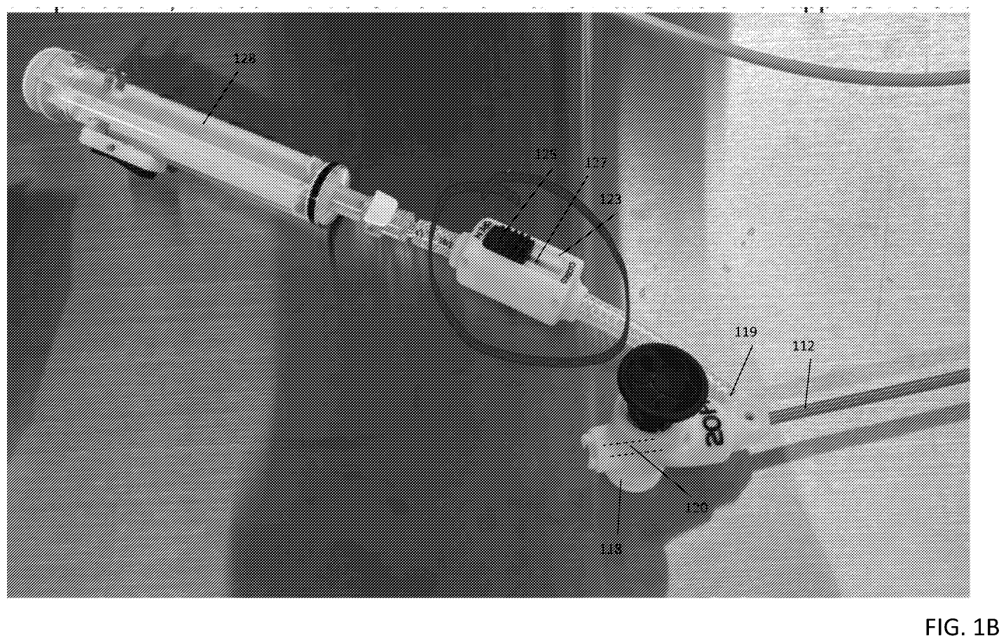

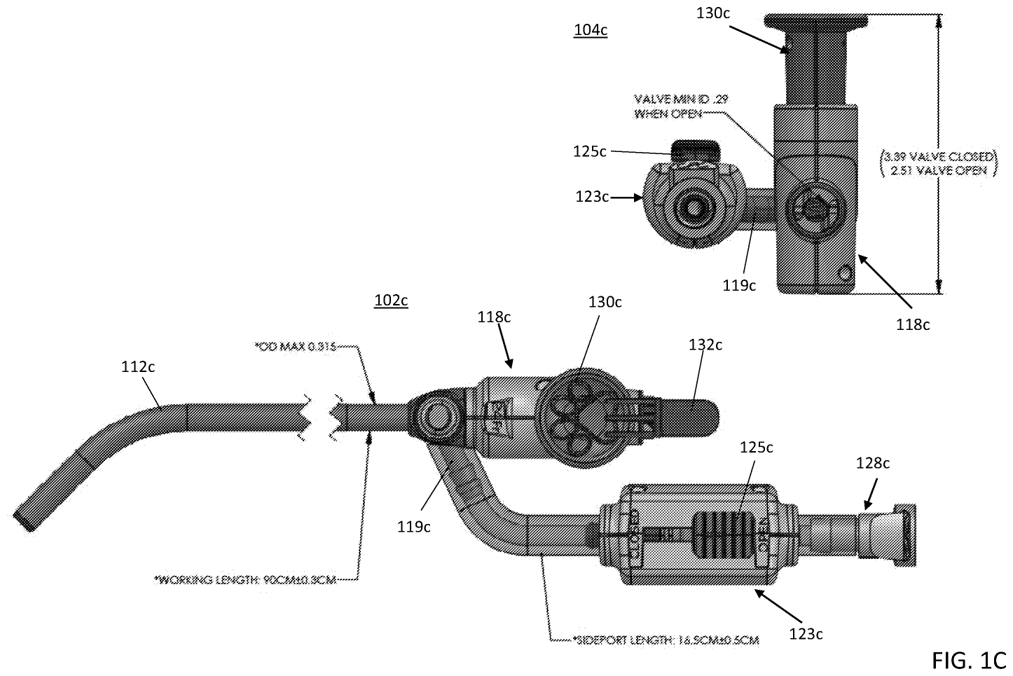

FIG. 1A illustrates a retrieval device 100, in accordance with some embodiments of the present specification. The device 100 comprises a first unit 102 that includes a handle 105 coupled to a tip portion 104 via a plurality of telescoping tubes, wherein the handle 105 is configured to steer the tip portion 104 in proximity to an occlusion to perform thrombectomy. In some embodiments, a proximal element 142 and a distal element 144 are positioned on the tip portion 104.

The device 100 further comprises a second unit 110 that includes an aspiration catheter 112 having a distal end 114 and a proximal end 116. The proximal end 116 is coupled to a port 118 which, in embodiments of the present specification, includes a flexible lumen tube (or a valve) 120. In embodiments, the device 100 includes an aspiration port 119 which is distal to flexible lumen tube or valve 120. A hub 122 is coupled to a proximal end 126 of the aspiration port 119. A negative pressure source 128, such as, for example, a syringe, is coupled to a proximal end 130 of the hub 122. Activating or deactivating a button 124 on the hub 122 enables suction (from the negative pressure source 128) to be applied to or isolated from the aspiration catheter 112 via the aspiration port 119.

During a thrombectomy procedure, the tip portion 104 is placed into a delivery catheter 135 and thereafter the delivery catheter 135 is inserted into the aspiration catheter 112 through the port 118, so that at least the tip portion 104 projects distally from the distal end 114 of the aspiration catheter 112. In accordance with aspects of the present specification, the device 100 is configured to enable an operator to single-handedly operate/actuate the handle portion 105 (using first, second and third knobs 150, 152 and 154) in order to mechanically extract occlusions, minimize tPA (tissue Plasminogen Activator), reduce bleeding, and aspirate by actuating the hub 122 while providing distal embolic protection.

In accordance with some aspects of the present specification, the first and second units 102, 110 are manufactured as separate standalone units or devices. This is advantageous in that a physician may use the first unit 102 with any third-party aspiration catheter. In some embodiments, the aspiration catheter 112 is available with a plurality of external diameters such as, but not limited to, 12 Fr, 16 Fr, 20 Fr, and 24 Fr (where Fr represents French scale or gauge system). In some embodiments, the syringe 128 has an exemplary, non-limiting, volume of 60 cubic centimeters.

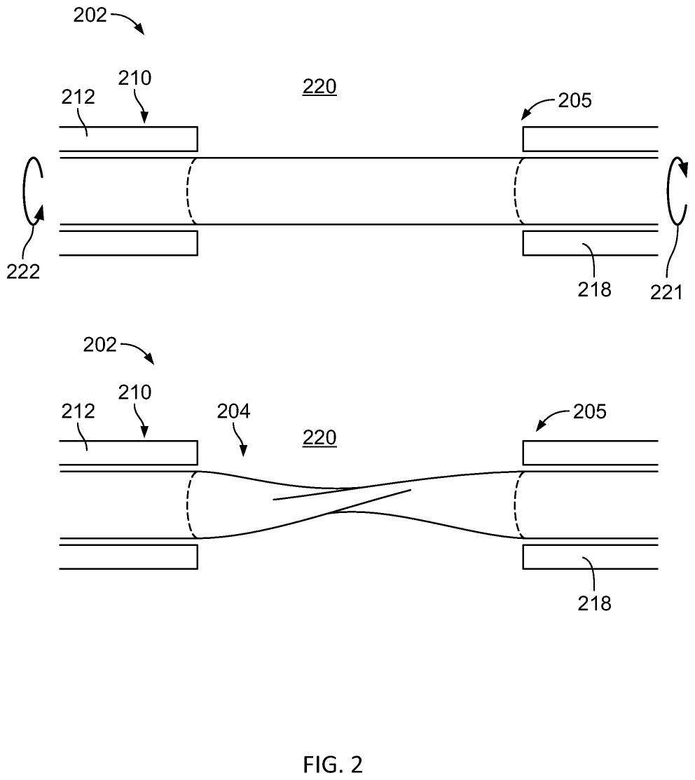

In some embodiments, apart from being used in mechanical thrombectomy procedures, hub and valve arrangements, described with reference to FIGS. 2, 3, 4, 5, 6 and 7 may also be used in large bore femoral closure procedures. More specifically, the thrombectomy device, in some embodiments, may be the devices disclosed in U.S. Pat. Nos. 10,172,634 and 10,898,215, U.S. patent application Ser. No. 17/127,521, and U.S. Patent Provisional Application Nos. 63/092,428 and 63/215,565, all of which are herein incorporated by reference in their entirety. The hub and valve embodiments may be positioned in fluid communication with catheter 112 by positioning it at flexible lumen tube or valve 120, wherein the distal portion of the hub and valve embodiments is in fluid communication with catheter 112 and wherein the proximal portion of the hub and valve embodiments is configured to receive medical instruments and allow blood to flow out.

FIG. 1B illustrates a photographic image of another embodiment of hub 118 coupled to catheter 112 and a valve 123 with a connected negative pressure source 128, in accordance with some embodiments of the present specification. Hub 118 includes flexible lumen tube or valve 120 positioned within the hub's body (shown with dashed lines in FIG. 1B). By default, the flexible lumen tube or valve 120 is in a constricted state such that any element cannot pass through it unless it is unconstricted. Catheter 112 is coupled to a distal end of the hub 118. Aspiration port 119 is included on hub 118, distal to the flexible lumen tube or valve 120 and proximal to the catheter 112. Suction may be applied to the aspiration port 119 distal to the flexible lumen tube or valve 120. Aspiration valve 123 is connected to the aspiration port 119 and is configured to control aspiration from the catheter 112. In some embodiments, the valve 123 is actuated by a thumb slider 125, which, when moved from an open position to a closed position, causes a member to crush down (or place pressure) on a soft tube 127 (also described herein as flexible lumen tube or valve 120) within the valve 123 to close the valve 123. In alternate embodiments, valve 123 is actuated using different mechanisms which are described below. The opening and closing of the valve 123 effectively applies aspiration from a negative pressure source 128, such as a syringe. Therefore, flexible lumen tube or valve 120 is brought to an unconstricted state for as long as the valve 123 is actuated, allowing for aspiration. The aspiration port 119 is positioned, and therefore aspiration occurs, distal to the flexible lumen tube or valve 120. Thus, in embodiments, the flexible lumen tube or valve 120 is not involved with aspiration, but the flexible lumen tube or valve 120 is used to seal to prevent leakage from its proximal end when suction is applied.

FIG. 1C illustrates another embodiment of a device assembly that includes hub 118c coupled to catheter 112c and a valve 123c with a connected negative pressure source 128c, in accordance with the present specification. A top view 102c of the assembly includes hub 118c and valve 123c, while a second view 104c of the assembly shows a profile view. In embodiments, hub 118c includes a flexible lumen tube or valve positioned within the hub's body (not shown). The valve, also termed herein as the hemostasis valve, is manipulated by an actuator 130c. In embodiments, and referring to profile view 104c, a profile length (height) of hub 118c ranges from 2 inches to 5 inches, and preferably 2.25 inches to 3.50 inches. Referring to profile view 104c, when hemostasis valve 130c is in a closed position, a profile length (height) of hub 118c is approximately 3.39 inches (in). When hemostasis valve 130c is in an open position, a profile length (height) of hub 118c is approximately 2.51 inches (in). In embodiments, catheter 112c is coupled to a distal end of hub 118c.

An aspiration port 119c is included on hub 118c, distal to the flexible lumen tube or valve and proximal to the catheter 112c. In some embodiments, a total length of aspiration port 119c from hub 118c to an aspiration valve 123c ranges between 10 cm and 20 cm and, is preferably in the range of 16 to 17 cm. Suction may be applied to aspiration port 119c distal to the flexible lumen tube or valve. Aspiration valve 123c is connected to aspiration port 119c and is configured to control aspiration from catheter 112c. In some embodiments, valve 123c is actuated by a thumb slider 125c, which, when moved from an open position (as shown in first view 102c) to a closed position, causes a member to crush down (or place pressure) on a soft tube (not shown) within valve 123c to close the valve 123c.

In embodiments, the opening and closing of the valve 123c is used to effectively apply aspiration from a negative pressure source 128c, such as a syringe which is connected to quick connector 126. The aspiration port 123c is positioned, and therefore aspiration occurs, distal to the flexible lumen tube or valve. Thus, in embodiments, the flexible lumen tube or hemostasis valve positioned within hub 118c is not involved with aspiration but is used to create a seal to prevent leakage from its proximal end when suction is applied. In other words, the flexible lumen tube or hemostasis valve, when in the first closed state, creates a seal laterally pressing against a previously introduced medical device to pass through catheter 112c, but the seal does not prevent axial or longitudinal movement of the device in the hub 118c and catheter 112c. In such a case, the seal continues to exert lateral pressure (normal to the surface of device) even as the device is longitudinally moved back and forth through the hub and catheter.

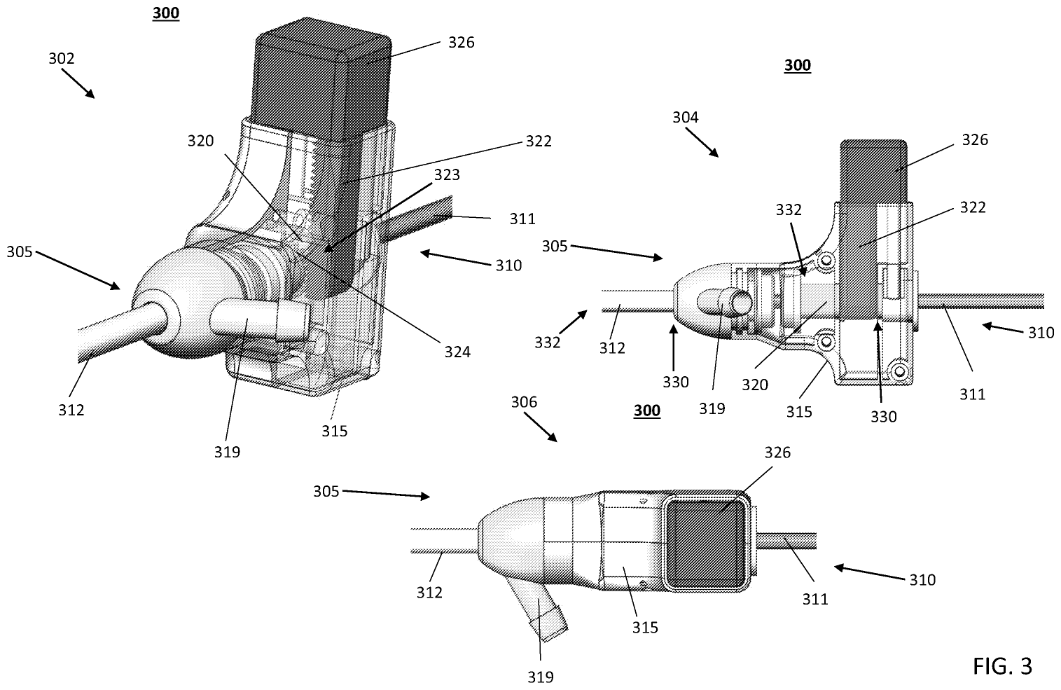

In some embodiments, the proximal and distal ends of the flexible lumen tube or hemostasis valve are spring loaded in a nominally twisted state causing the lumen of the tube or hemostasis valve to be constricted or pinched and to require a force to be applied to the spring to untwist the lumen of the valve and thereby unconstrict or unpinch the lumen. In some embodiments, in the unconstructed or open state, the hemostasis valve has a minimum ID of 0.29 cm. In some embodiments, the hemostasis valve is made by adding a thermoplastic polyurethane, which includes an ultra-soft polyether or polyester-based blend (such as, but not limited to, NeuSoft UR842A) to the design that shrouds the ePTFE for additional robustness to enhance sealing. In some embodiments, a cylindrical cap 132c is fixedly attached to actuator 130c of hemostasis valve. Cap 132c, made from plastic in an embodiment, depresses the top of actuator 130c to remove twisting strain and keep the hemostasis valve open during the time when hub 118c is kept on a shelf before its use. At the time of deployment, user may remove cap 132c by pulling it out to detach it from actuator 130c. More details of hub 118c with hemostasis valve and actuator are described below with reference to FIG. 3.

FIG. 2 illustrates first and second cross-sectional views 202, 204 of a flexible lumen tube or hemostasis valve 220 (valve 120 in FIG. 1A), in accordance with some embodiments of the present specification. View 202 illustrates the flexible lumen tube or valve 220 in a second state while view 204 illustrates the flexible lumen tube or valve 220 in a first state. In embodiments, valve 220 stays in the first state in its original or default condition. Further, valve 220 remains in the second state for as long as an actuator connected to valve 220 is activated. The second state corresponds to the flexible lumen tube or valve 220 being open or untwisted while the first state corresponds to the flexible lumen tube or valve 220 being constricted, pinched or twisted.

The flexible lumen tube or valve 220 has proximal and distal ends 205, 210. The proximal end 205 is coupled to a hub 218 (hub 118 in FIG. 1A) while the distal end 210 is coupled to an aspiration catheter 212 (catheter 112 in FIG. 1A). As shown in the second view 204, in some embodiments, the proximal and distal ends 205, 210 are spring loaded in a nominally twisted or first state causing the lumen of the tube of valve 220 to be constricted or pinched.

As shown in the first view 202, by rotating the proximal and distal ends 205, 210 relative to each other, the flexible lumen tube or valve 220 untwists so that the lumen of the tube or valve 220 is open or in the second state. In some embodiments, the proximal end 205 may be rotated in a first direction (e.g., clockwise or counterclockwise) and the distal end 210 in a second direction (e.g. counterclockwise or clockwise) opposite to the first causing the flexible lumen tube or valve 220 to untwist so that the lumen of the tube or valve 220 is open or in the second state. In some embodiments, the distal end 210 is fixed while the proximal end 205 is rotated causing the flexible lumen tube or valve 220 to untwist so that the lumen of the tube or valve 220 is open or in the second state. When in the closed or first (default) state, as shown in second view 204, the twisted flexible lumen tube or valve 220 of the catheter hub seals the proximal end of the catheter 212. When in the open or second state, shown in first view 202, the flexible lumen tube or valve 220 allows medical devices to pass through and blood to flow out.

In some embodiments, the flexible lumen tube or valve 220 is made of thin flexible materials such as, but not limited to, PTFE (polytetrafluoroethylene), ePTFE (expanded polytetrafluoroethylene), urethane or silicone. In some embodiments, the tube or valve 220 may be reinforced with stainless, NiTi (Nitinol) and/or nylon members, or NeuSoft UR842A. In some embodiments, the flexible lumen tube or valve 220 has an inner diameter ranging from 0.01 inch to 1 inch. In some embodiments, the flexible lumen tube or valve 220 has an inner diameter ranging from 0.01 inch to 2 inches. In some embodiments, a thickness of the wall of the flexible lumen tube or valve 220 ranges from 0.0002 inch to 0.125 inch. In some embodiments, a length of the flexible lumen tube or valve 220 ranges from 0.01 inch to 2 inches. In some embodiments, a length of the flexible lumen tube or valve 220 is at least equal to a diameter of the flexible lumen tube or valve to achieve a fully closed configuration by twisting, and thereby a reliable constriction. In some embodiments, a diameter of the flexible lumen tube or valve 220 is 0.47 inches (12 mm) and a length of the flexible lumen tube or valve 220 is 0.47 inches (12 mm). In various embodiments, the flexible lumen tube or valve 220 is configured to rotate angularly ranging from 90 degrees to 360 degrees in order to seal, constrict or pinch the valve 120. In some embodiments, the flexible lumen tube or valve 220 is configured to seal against pulmonary artery pressure ranging from 0 French to 24 French.

In various embodiments, the hub 218 (hub 118 in FIG. 1A) is actuated by a rotational or twisting motion in a first direction 221 to enable the proximal and distal ends 205, 210 to rotate relative to each other, thereby causing the flexible lumen tube or valve 220 to untwist so that the lumen of the tube or valve 220 is open or in the second state. Flexible lumen tube or valve 220 remains in the second state for the duration of actuation of hub 218. In embodiments, the catheter 212 may be held securely relative to hub 218, or rotated or twisted in a second direction 222 opposite to the first direction 221, to assist in opening the lumen of the tube or valve 220.

FIG. 3 shows perspective, side and top views 302, 304, 306 of a hub 300, including a single rack and pinion arrangement 323, for use with a flexible lumen tube or valve 320, in accordance with some embodiments of the present specification. In embodiments, the hub 300 is coupled to the aspiration catheter 312 at a distal end 305 of the hub 300 and is configured to receive a medical device 311 at a proximal end 310 of the hub 300. The flexible lumen tube or valve 320 has a proximal end 330 that is free to rotate and a distal end 332 fixedly coupled to aspiration catheter 312, that is positioned within the hub 300, and is configured to be changed from a first twisted or constricted state to a second open or unconstricted state by the rack and pinion arrangement 323. In embodiments, by default, the flexible lumen tube or valve 320 is in a twisted state causing the lumen of the tube or valve 320 to be constricted or pinched, thereby preventing any blood to flow out through the hub 300 via the flexible lumen tube or valve 320 or the introduction of a medical device 311 into the hub 300 and catheter 312 via the flexible lumen tube or valve 320. In some embodiments, a medical device 311 previously passed through the hub 300 and into the catheter 312 may still be moved even when the flexible lumen tube or valve 320 is in the closed first state. In other words, the flexible lumen tube or valve 320, when in the first closed state, creates a seal laterally pressing against the previously introduced medical device 311, but the seal does not prevent axial or longitudinal movement of the device 311 in the hub 300 and catheter 312. In such a case, the seal continues to exert lateral pressure (normal to the surface of device) even as the device is longitudinally moved back and forth through the hub and catheter. In some embodiments, proximal and distal ends 330, 332 of the flexible lumen tube or valve 320 are spring loaded in a nominally twisted state causing the lumen of the tube of valve 320 to be constricted or pinched and to require a force to be applied to the spring to untwist the lumen of the valve and thereby unconstrict or unpinch the lumen. An aspiration port 319 is included at the distal end of the hub 300 for connection to a negative pressure source (128 in FIG. 1A), such as, for example, a syringe.

As shown, the hub 300 has a body or housing 315 that houses a rack and pinion arrangement 323, comprising a rack 322 and associated pinion or gear 324 mechanism. A button or knob 326 is positioned on the body or housing 315. The button or knob 326 is coupled to the rack 322 which in turn is engaged with the associated pinion or gear 324. The pinion or gear 324 is coupled to the proximal end 330 of the flexible lumen tube or valve 320. The pinion 324 moves laterally by a spring that pushes a proximal pinioned valve module away from the distal valve module, so that when the button 326 is pushed, the proximal pinioned module rotates, opening the flexible lumen tube or valve 320 and freeing up slack in the valve. The slack is taken up by the proximal pinioned valve module being pushed by the spring away from the distal module, lengthening the valve 320, keeping it in tension.

Pressing or depressing of the button or knob 326 downwards causes the rack 322 to slide, translate or move downwards and cause the associated pinion or gear 324 to rotate in a first direction. Rotation of the pinion or gear 324 causes the proximal end 330 of the flexible lumen tube or valve 320 to also rotate (since the pinion or gear 324 is coupled to the proximal end 330) relative to the distal end 332 of the flexible lumen tube or valve 320. This rotation of the proximal and distal ends 330, 332 relative to each other causes the flexible lumen tube or valve 320 to untwist so that the lumen of the tube or valve 320 is in an open state. In the open state, the flexible lumen tube or valve 320 allows passage of a medical device into the hub 305 and catheter 312 or blood flow out of the catheter 312.

Releasing of the button or knob 326 (as a result of which the button or knob 326 moves upwards) causes the rack 322 to slide, translate or move back upwards (to its original position) and cause the associated pinion or gear 324 to rotate in a second direction opposite the first direction. Rotation of the pinion or gear 324 causes the proximal end 330 of the flexible lumen tube or valve 320 to also rotate (since the pinion or gear 324 is coupled to the proximal end 330) relative to the distal end 332 of the flexible lumen tube or valve 320. This rotation of the proximal and distal ends 330, 332 relative to each other, in the second direction, causes the flexible lumen tube or valve 320 to twist again causing the lumen of the tube or valve 320 to be constricted or pinched thereby sealing the proximal end 330 of the flexible lumen tube or valve 320. Thus, the applied pressure on the button or knob 326 is translated, by the rack 322, to rotation motion of the pinion or gear 324.

In some embodiments, the upwards movement of the rack 322 and the rotation of the pinion or gear 324 in the second direction is aided by the spring load effective on the proximal and distal ends 330, 332 of the flexible lumen tube or valve 320.

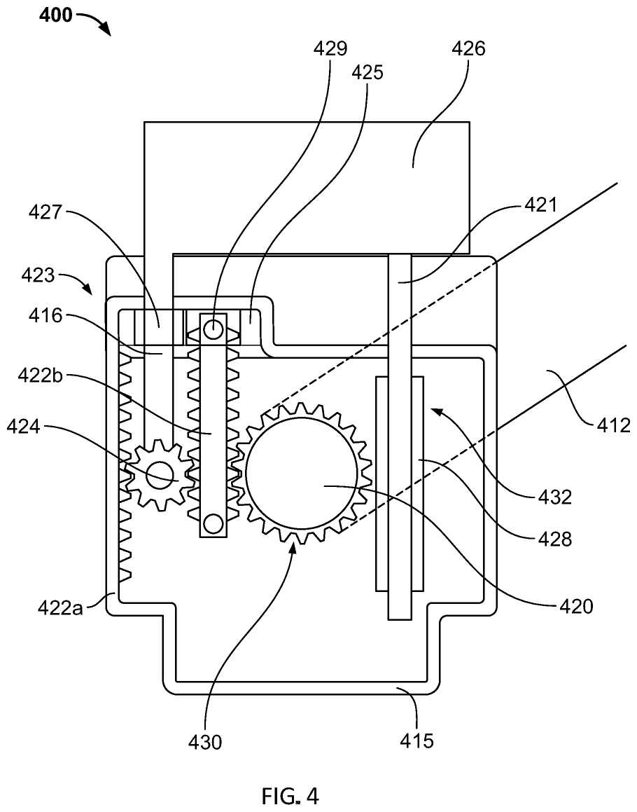

FIG. 4 shows a front-on view of a proximal end of a hub 400 including a double rack and pinion arrangement 423, for use with a flexible lumen tube or valve 420, in accordance with some embodiments of the present specification. In embodiments, the hub 400 includes the flexible lumen tube or valve 420 within. A catheter 412 is coupled to the distal end of the flexible lumen tube or valve 420. The flexible lumen tube or valve 420 has a proximal end 430 that is free to rotate and a distal end 432 that is fixedly coupled to an aspiration catheter 412. In embodiments, by default, the flexible lumen tube or valve 420 is in a twisted state causing the lumen of the tube or valve 420 to be constricted or pinched thereby sealing the flexible lumen tube or valve 420. In some embodiments, the proximal and distal ends 430, 432 are spring loaded in a nominally twisted state causing the lumen of the tube of valve 420 to be constricted or pinched.

As shown, the hub 400 has a body or housing 415 that houses a first rack 422a, a second rack 422b and a pinion or gear 424 engaged with the first and second racks 422a, 422b. In some embodiments, the first rack 422a is stationary while the second rack 422b is slidable or movable. A button or knob 426 is positioned on the body or housing 415. The button or knob 426 is coupled to the pinion or gear 424 by a slidable arm 416 extending from a bottom surface of the button or knob 426 and the second rack 422b (which is slidable) is coupled to the proximal end 430 of the flexible lumen tube or valve 420. In some embodiments, the double rack and pinion arrangement 423 includes a horizontal support member 425 positioned within the body or housing 415. The horizontal support member 425 includes a first opening 427 and a second opening 429. The first opening 427 is configured to slidably receive the arm 416 and the second opening 429 is configured to slidably receive the second rack 422b. When the button or knob 426 is pressed, the horizontal support member 425 allows up and down movement of the arm 416 and the second rack 422b, through the first opening 427 and the second opening 429 respectively, while preventing lateral movement of these elements, thereby stabilizing the double rack and pinion arrangement 423 in the hub 400. In embodiments, a vertical support member 421 extends from the bottom surface of the button or knob 426 and is configured to move into and out of a recess 428 positioned within the body or housing 415 as the button or knob 426 is pressed. The vertical support member 421 and recess 428 function as a guide support for the button or knob 426 and serve to stabilize the hub 400 and double rack and pinion arrangement 423.

Pressing or depressing of the button or knob 426 downwards causes the pinion or gear 424 to rotate and also move axially, in a first direction, along the first rack 422a which is stationary. Additionally, the rotation and axial movement of pinion or gear 424 causes the second rack 422b to move or slide in the first direction. The sliding movement of the second rack 422b causes the proximal end 430 of the flexible lumen tube or valve 420 to also rotate in the first direction (since the second rack 422b is coupled to the proximal end 430) relative to the distal end 432 of the flexible lumen tube or valve 420. This rotation of the proximal and distal ends 430, 432 relative to each other, in the first direction, causes the flexible lumen tube or valve 420 to untwist so that the lumen of the tube or valve 420 is in an open state. In the open state, the flexible lumen tube or valve 420 enables the flow of blood or passage of a medical device.

It should be appreciated that the second rack 422b moves twice as far as the button or knob 426 since it is acted upon by the axial movement as well as the rotational motion of the pinion or gear 424. In other words, the double rack and pinion system of the hub 400 amplifies the stroke of the button or knob 426 allowing for less button or knob 426 travel to achieve the same angular rotation of the proximal end 430 of the flexible lumen tube or valve 420, relative to an embodiment with a single rack (for example, the single rack and pinion arrangement 323 depicted in FIG. 3). In some embodiments, the double rack and pinion system of the hub 400 amplifies the stroke of the button or knob 426 at a rate of 2:1 relative to an embodiment with a single rack.

Releasing of the button or knob 426 (as a result of which the button or knob 426 moves upwards) causes the pinion or gear 424 to rotate and also move axially, in a second direction opposite to the first direction, along the first rack 422a (which is stationary). Additionally, the rotating and axially moving pinion or gear 424 causes the second rack 422b to move or slide in the second direction. The sliding movement of the second rack 422b causes the proximal end 430 of the flexible lumen tube or valve 420 to also rotate in the second direction (since the second rack 422b is coupled to the proximal end 430) relative to the distal end 432 of the flexible lumen tube or valve 420. This rotation of the proximal and distal ends 430, 432 relative to each other, in the second direction, causes the flexible lumen tube or valve 420 to twist again causing the lumen of the tube or valve 420 to be constricted or pinched thereby preventing the flow of blood or passage of a medical device through the flexible lumen tube or valve.

In some embodiments, the movement of the second rack 422b in the second direction and that of the pinion or gear 424 in the second direction is aided by the spring load effective on the proximal and distal ends 430, 432 of the flexible lumen tube or valve 420.

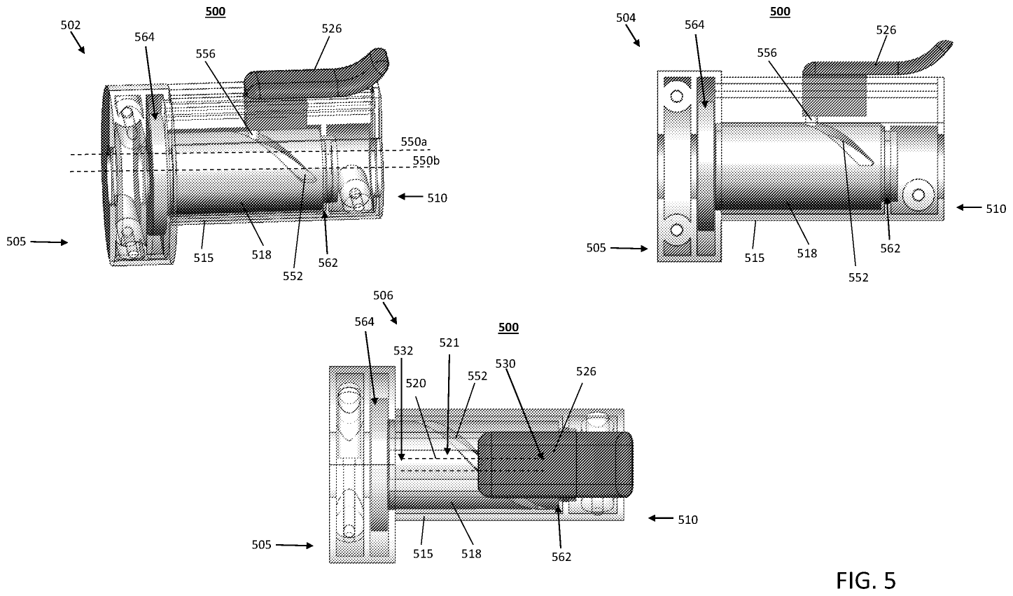

FIG. 5 shows perspective, side and top views 502, 504, 506 of a hub 500 including a twist mechanism driven by a cammed slider, for use with a flexible lumen tube or valve 520, in accordance with some embodiments of the present specification. In embodiments, the hub 500 includes flexible lumen tube or valve 520 within. The flexible lumen tube or valve 520 has a proximal end 530 that is free to rotate and a distal end 532 that is fixedly coupled to an aspiration catheter. In embodiments, by default, the flexible lumen tube or valve 520 is in a twisted state causing the lumen of the tube or valve 520 to be constricted or pinched thereby preventing the flow of blood or passage of a medical device through the flexible lumen tube or valve 520 from or to the aspiration catheter. In some embodiments, the proximal and distal ends 530, 532 are spring loaded in a nominally twisted state causing the lumen of the tube of valve 520 to be constricted or pinched.

As shown, the hub 500 has a body or housing 515 that houses an inner element 518. In some embodiments, the inner element 518 has a substantially cylindrical shape. The inner cylindrical element 518 houses the flexible lumen tube or valve 520 (shown with dotted lines). The cylindrical inner element 518 has a predefined outer diameter, a proximal end 562 and a distal end 564. As shown in the view 502, a longitudinal axis 550a of the cylindrical inner element 518 lies substantially parallel to a longitudinal axis 550b of the hub 500. A substantially helical groove 552 is formed on an outer surface of the inner element 518 such that the helical groove 552 wraps around the outer diameter of the inner element 518.

A slider 526 is positioned on the body or housing 515. The slider 526 is constrained to move axially, substantially parallel to the longitudinal axis 550b of the hub 500, within a slot (of a predefined length) 521 formed in the body or housing 515. A pin 556 attached to the slider 526 engages with and tracks the groove 552.

The proximal end 562 of the inner element 518 is in fluidic communication with the proximal end 510 of the hub 500 while the distal end 564 of the inner element 518 is in fluid communication with the distal end 505 of the hub 500.

Moving or translating the slider 526 distally or forward causes the pin 556 to move within the substantially helical groove 552 thereby rotating the inner element 518 in a first direction. Thus, the inner element 518 rotates based on the position of the pin 556 in the groove 552 and distance travelled by the slider 526 in the slot 521. Rotation of the inner element 518 causes the proximal end 530 of the flexible lumen tube or valve 520 to also rotate (since proximal end 562 of the inner element 518 is coupled to the proximal end 530 of the flexible lumen tube or valve 520) relative to the distal end 532 of the flexible lumen tube or valve 520. This rotation of the proximal and distal ends 530, 532 relative to each other causes the flexible lumen tube or valve 520 to untwist so that the lumen of the tube or valve 520 is in an open state. In the open state, the flexible lumen tube or valve 520 allows blood to flow or a medical device to be passed through the hub 500 via the flexible lumen tube or valve 520.

Moving or translating the slider 526 proximally or backward away from the flexible lumen tube or valve 520 causes the pin 556 to move within the substantially helical groove 552 thereby rotating the inner element 518 in a second direction opposite to the first direction. Rotation of the inner element 518 causes the proximal end 530 of the flexible lumen tube or valve 520 to also rotate (since proximal end 562 of the inner element 518 is coupled to the proximal end 530 of the flexible lumen tube or valve 520) relative to the distal end 532 of the flexible lumen tube or valve 520. This rotation of the proximal and distal ends 530, 532 relative to each other, in the second direction, causes the flexible lumen tube or valve 520 to twist again causing the lumen of the tube or valve 520 to be constricted or pinched thereby sealing the flexible lumen tube or valve 520 and preventing the flow of blood or passage of a medical device through the aspiration catheter.

In some embodiments, the backward movement of the slider 526 and the rotation of the inner element 518 in the second direction is aided by the spring load effective on the proximal and distal ends 530, 532 of the flexible lumen tube or valve 520.

In accordance with aspects of the present specification, the hubs of FIGS. 3, 4 and 5 are characterized by buttons, knobs or sliders that are configured to be actuated or manipulated single-handedly by a user in order to control the flexible lumen tube or valve and therefore application and prevention of suction or negative pressure through the flexible lumen tube or valve.

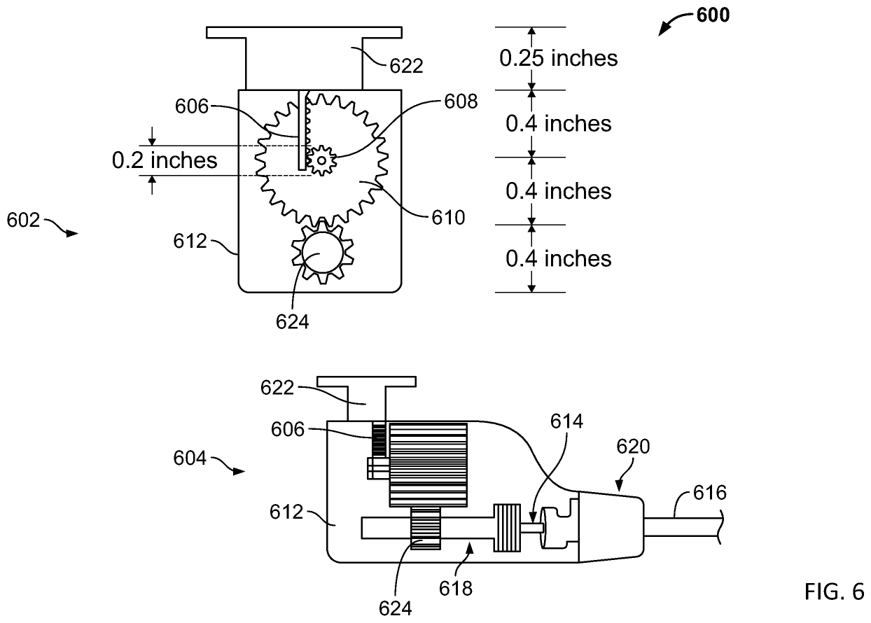

FIG. 6 illustrates another embodiment of a hub included in a valve mechanism, in accordance with the present specification. A first view 602 is a schematic illustration of a front view of a valve mechanism 600. A second view 604 is a schematic illustration of a side view of valve mechanism 600 with a proximal side on the left and a distal side on the right. Referring simultaneously to views 602 and 604, the schematics illustrate an arrangement of a rack 606, a pinion 608, and a stepped-up gear 610 within a housing 612. The arrangement is used to operate both constriction/closing and expansion/opening of a flexible lumen tube or valve 614, in accordance with some embodiments of the present specification.

In embodiments, flexible lumen tube or valve 614 is placed within housing 612 of hub 600. An aspiration catheter 616 is coupled to the distal end of the flexible lumen tube or valve 614. The flexible lumen tube or valve 614 has a proximal end 618 that rotates freely and a distal end 620 that is fixedly coupled to aspiration catheter 616. In embodiments, by default, the flexible lumen tube or valve 614 is in a first twisted state causing the lumen of the tube or valve 614 to be constricted or pinched thereby sealing the flexible lumen tube or valve 614. In some embodiments, the proximal and distal ends 618, 620 are spring loaded in a nominally twisted state which causes constriction or pinching of the lumen of the tube of valve 614.

As shown, the hub 600 has a body or housing 612 that houses rack 606 and a pinion or gear 608 configured to engage with a proximal end of rack 606. Additionally, pinion 608 is configured to engage with a stepped-up gear 610 having a radius that is in a range of 2 to 6 times, preferably 4 times, the radius of the pinion 608. Rack 606 extends linearly along a tangent to pinion 608, with a distal end connected to an actuator 622. In embodiments, actuator 622 is a press-button or a knob with a height of approximately 0.25 inch and is positioned on top of housing 612. When actuator 622 is pressed, rack 606 (by way of its connection to actuator 622) is linearly displaced towards a bottom side of housing 612, causing pinion 608 to rotate in one direction. Activation of actuator 622 (in an embodiment, by pressing or depressing downwards) causes pinion 608 to rotate and also move axially, in a first direction, along a length of rack 606. Additionally, as pinion 608 rotates, the rotation and axial movement of pinion 608 causes the stepped-up gear 610, which is configured to engage with pinion 608, to rotate in the first direction. Stepped-up gear 610 is coupled to a proximal end 618 of the flexible lumen tube or valve 614. In some embodiments, stepped-up gear 610 is coupled to the flexible lumen tube or valve 614 through a gear 624 positioned at proximal end 618. In an embodiment, gear 624 has a radius that is approximately ¾ to ⅙ the radius of stepped up gear 610, and preferably ½ the radius of stepped up gear 610. In embodiments, radius of gear 624 is as small as possible to achieve the most rotation with press of actuator 622, while allowing proximal end 618 of the flexible lumen tube or valve 614 to pass through it.

The rotation of stepped-up gear 610 causes proximal end 618 of the flexible lumen tube or valve 614 to also rotate relative to distal end 620. It should be appreciated that stepped-up gear configuration provides greater rotation, or rotation at a faster rate than the actuation or pressing of the actuator, of the proximal end 618 of the flexible lumen tube or valve 614 relative to distal end 620. In other words, the stepped-up gear 610 configuration of the hub 600 amplifies the stroke of the actuator 622, allowing for actuator 622 travel to achieve the same angular rotation of the proximal end 618 of the flexible lumen tube or valve 614, relative to an embodiment with a single gear (for example, the single rack and pinion arrangement 323 depicted in FIG. 3). In some embodiments, the stepped-up gear 610 configuration of the hub 600 amplifies the stroke of the actuator 622 at a rate of 2:1 relative to an embodiment with a single gear. This relative rotation of the proximal end 618 and distal end 620, causes the flexible lumen tube or valve 614 to untwist or straighten in a third direction so that the lumen of the tube or valve 614 is in an open state. In the open state, the flexible lumen tube or valve 614 enables the flow of blood or passage of a medical device through catheter 616.

Releasing of actuator 622 (as a result of which the button or knob moves upwards) causes pinion 608 to rotate and also move axially, in a second direction opposite to the first direction, along rack 606. In turn, the rotation and axial movement of pinion 608 causes stepped-up gear 610 to rotate in the second direction. Finally, the rotation of stepped-up gear 610 causes the proximal end 618 of the flexible lumen tube or valve 614 to also rotate relative to distal end 620. The relative rotation of the proximal end 618 and distal end 620, in a fourth direction opposite to the third direction, causes the flexible lumen tube or valve 614 to twist again causing the lumen of the tube or valve 614 to be constricted or pinched thereby preventing the flow of blood or passage of a medical device through the flexible lumen tube or valve.

In some embodiments, the movement of both pinion 608 and stepped-up gear 610 in the second direction is aided by the spring load effective on the proximal end 618 and distal end 620 of the flexible lumen tube or valve 614.

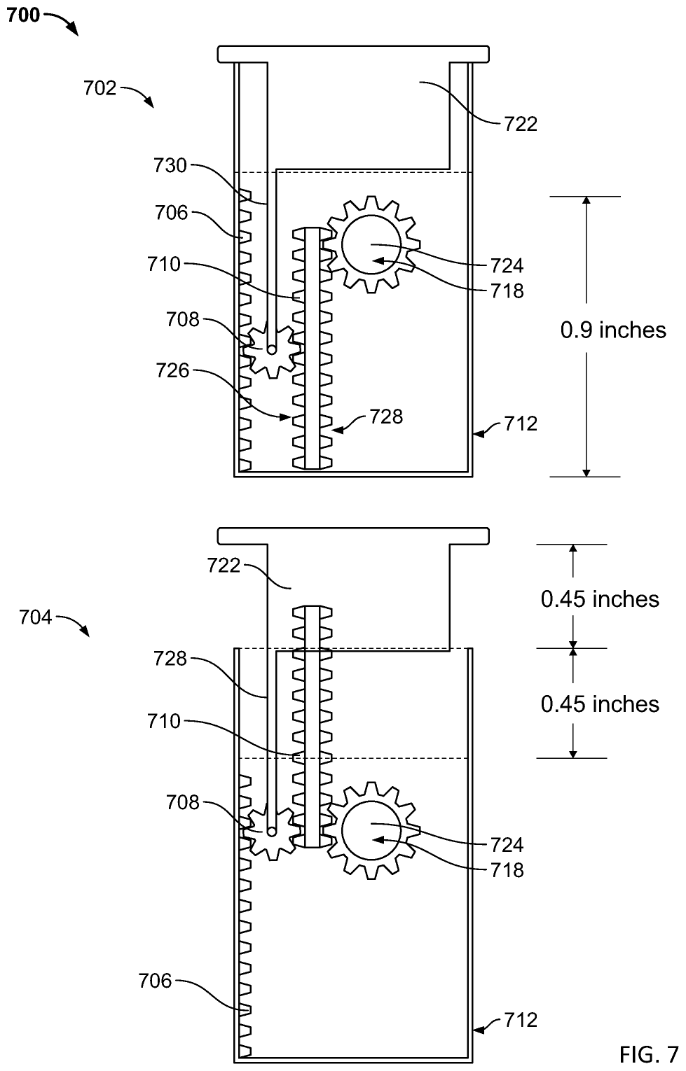

FIG. 7 illustrates another embodiment of a hub 700 included in a valve mechanism, in accordance with the present specification. A first view 702 is a schematic cross-sectional view of a valve mechanism 700 when an actuator 722 is pressed. A second view 704 is a schematic cross-sectional view when actuator 722 is released and/or is not pressed. Referring simultaneously to views 702 and 704, the schematic diagrams illustrate a configuration of a first fixed rack 706, a pinion 708, and a second floating rack 710 within a housing 712. In accordance with some embodiments, the configuration is employed to enable constriction/closing and expansion/opening of a flexible lumen tube or valve (not shown).

In embodiments, hub 700 includes a housing 712 within which the flexible lumen tube or valve (not shown) is placed. An aspiration catheter (not shown) is coupled to the distal end of the flexible lumen tube or valve. The flexible lumen tube or valve has a proximal end that rotates freely and a distal end that is fixedly coupled to aspiration catheter. In embodiments, by default, the flexible lumen tube or valve is in a first twisted state causing the lumen of the tube or valve to be constricted or pinched thereby sealing the flexible lumen tube or valve. In some embodiments, the proximal end and distal end are spring loaded in a nominally twisted state causing the lumen of the tube of valve to be constricted or pinched.

In embodiments, a fixed rack 706 is positioned along a vertical linear axis on an internal wall of housing 712. A jagged or toothed edge of fixed rack 706 faces internally within housing 712. Pinion 708 is configured to engage with fixed rack 706. Floating rack 710, which has a first side 726 and a second side 728, is positioned parallel to fixed rack 706 is configured to engage with pinion 708 on its first side 726. Floating rack 710, on its second side 728 (opposite to the first side 726), is configured to engage with a gear 724. In an embodiment, floating rack 710 has a length in a range of 0.3 to 2 inches, preferably approximately 0.9 inches, which is available to pinion 708 during its lateral movement. Gear 724 comprises a portion of a proximal side 718 of a valve mechanism, which further comprises the flexible lumen tube or valve. The relative configuration of gear 724 and the flexible lumen tube or valve is similar to that described in FIG. 6 and is not repeated herein.

Further, pinion 708 is attached to a bottom side of actuator 722 through an elongated connector 730. In embodiments, actuator 722 is a press-button or a knob and is positioned on top of housing 712. When actuator 722 is pressed from its top side, actuator 722 is moves downward, as shown in view 702. Connector 730, which is attached to a bottom side of actuator 722, then moves in a downward direction, causing pinion 708 to also move in the downward direction and simultaneously rotate along racks 706 and 710. Additionally, the rotation and axial movement of pinion 708 causes floating rack 710 to move linearly in a downward direction. The movement of floating rack 710 causes gear 724 to rotate and results in the proximal end 718 of the flexible lumen tube or valve to also rotate relative to the distal end. This relative rotation of the proximal end 718 and the distal end causes the flexible lumen tube or valve to untwist so that the lumen of the tube or valve is in an open state. In the open state, the flexible lumen tube or valve enables the flow of blood or passage of a medical device through the aspiration catheter.

It should be appreciated that pinion 708 is both moving and spinning around its axis, so actuator 722 moves twice the distance during a stroke of the button. In an embodiment, actuator 722 that has a length in a range of 0.3 to 2 inches, preferably approximately 0.9 inches, has bottom half of its length within housing 712 while the top half remains above and outside housing 712, when actuator 722 is pressed down. Therefore, system 700 amplifies the stroke of actuator 722 allowing for less amount of displacement of actuator 722 to achieve the same angular rotation of gear 724 and thus that of the flexible lumen tube or valve.

Releasing of actuator 722 (as a result of which the button or knob moves upwards) causes pinion 708 to rotate and also move axially, in an upward direction opposite to the downward direction, along rack 706, as shown in view 704. Additionally, the rotation and axial movement of pinion 708 causes floating rack 710 to move in the upward direction. The rotation of rack 710 causes the proximal end 718 of the flexible lumen tube or valve to also rotate relative to the distal end. The relative rotation of the proximal and distal ends, in a direction opposite to the previous direction, causes the flexible lumen tube or valve to twist again causing the lumen of the tube or valve to be constricted or pinched thereby preventing the flow of blood or passage of a medical device through the flexible lumen tube or valve. In some embodiments, the movement of rack 710 in the upward direction and that of pinion 708 is aided by the spring load effective on the proximal end 718 and the distal end of the flexible lumen tube or valve.

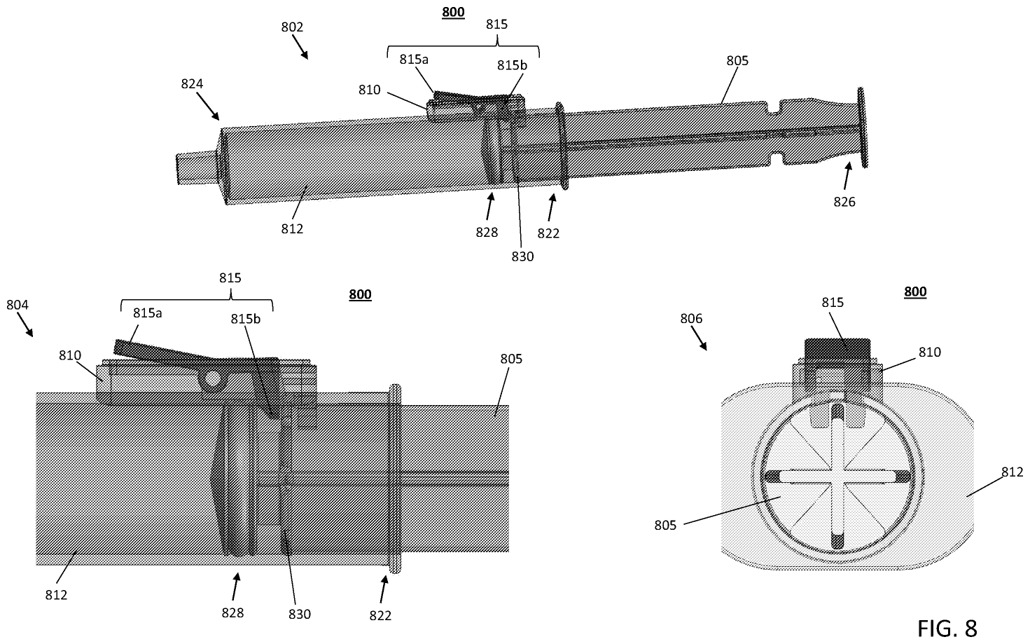

FIG. 8 shows first, second and third views 802, 804, 806 of a locking syringe 800, previously shown as element 128, in accordance with some embodiments of the present specification. In various embodiments, the locking syringe 800 is used as a negative pressure or suction source that is applied to an aspiration catheter through an aspiration port of a hub (119 in FIG. 1A). In accordance with aspects of the present specification, the syringe 800 is configured to automatically hold a plunger 805 in a drawn back state until released by a user.

Referring now to the views 802, 804, 806, the syringe 800 includes the plunger 805, a base 810 of a latch attached to on an outer surface of a syringe barrel or body 812 and a lever 815 movably coupled to the base 810. The lever 815 is pivoted relative to the base 810 and includes an arm 815a at one end and a hammer 815b at the other end. The syringe barrel or body 812 has a proximal end 822 and a distal end 824 while the plunger 805 has a proximal end 826 and a distal end 828. In some embodiments, the base 810 of the latch is positioned proximate the proximal end 822 of the syringe barrel or body 812. The distal end 828 of the plunger 805 includes a flange or ledge 830.

As shown in views 802 and 804, when the plunger 805 is drawn back or proximally the hammer 815b falls into place distal to or behind the flange or ledge 830 thereby preventing the plunger 805 to be pushed forward or distally. In some embodiments, the lever 815 is biased (such as, for example, by a spring load) at the pivot to cause the hammer 815b to fall into place by default. When the arm 815a of the lever 815 is depressed, the hammer 815b is pulled up (since the lever 815 is pivoted relative to the base 810) thereby releasing the plunger 805 to be pushed forward or distally.

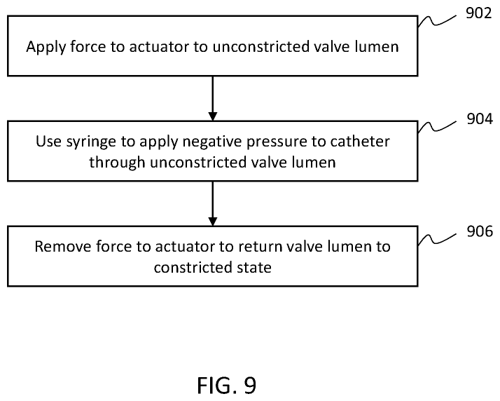

FIG. 9 is a flow chart illustrating an exemplary method for effectuating or causing aspiration using an aspiration catheter connected to an aspiration port of a hub with a locking syringe (such as syringe 800 of FIG. 8), in accordance with some embodiments of the present specification. In its original or default state, a valve comprising a flexible lumen is coupled to the aspiration catheter. The valve is held by a mechanism within the hub such that the lumen inside the valve is in a twisted configuration that constricts the lumen provides a seal. The mechanism positioned within the hub may be any one the various embodiments described above in the present specification. At step 902, a user applies force to an actuator to engage the mechanism within the hub in such a way that the valve lumen is untwisted in a first direction to an unconstricted state. The lumen stays in this unconstricted state for as long as the force is applied to the actuator.

At step 904, the user uses locking syringe connected to the hub to apply negative pressure through the unconstricted valve lumen to the catheter. The syringe is configured to hold a plunger in a drawn back state until released. The default position of plunger in its held-back state provides negative pressure for aspiration when the valve lumen is in an unconstricted state. When the plunger is drawn back the hammer (hammer 815b of FIG. 8) falls into place distal to or behind the ledge (ledge 830 of FIG. 8) thereby preventing the plunger to be pushed forward or distally. A lever is movably coupled to a base of a latch attached on an outer surface of syringe's barrel. The lever is biased (such as, for example, by a spring load) at a pivot to cause the hammer to fall into place by default. Only when an arm of the lever is depressed, the hammer is pulled up (since the lever is pivoted relative to the base) thereby releasing the plunger to be pushed forward or distally.

At step 906, the user removes the applied force to the actuator to return the valve lumen in its original twisted (constricted) state.

The above examples are merely illustrative of the many applications of the devices of the present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A valve system configured to be coupled to a catheter, the valve system comprising:
a valve having a proximal end and a distal end, wherein the distal end of the valve is coupled to a lumen of the catheter, wherein the valve comprises a flexible material extending between the proximal end and distal end and wherein the valve lumen has a first twisted state and a second untwisted state; and
a hub having a body, a rack, a pinion physically coupled to the rack, and an actuator, wherein the body encloses the rack, wherein the pinion is coupled to the proximal end of the valve, and wherein the actuator is coupled to the rack such that applying a force to the actuator moves the rack which causes the pinion to rotate in a first direction and thereby causes the lumen to untwist and adopt the second untwisted state.

2. The valve system of claim 1, wherein the actuator is coupled to the rack such that removing a pressure or physical force from the actuator automatically causes the rack to move and thereby rotate the pinion in a second direction to cause the lumen to adopt the first twisted state.

3. The valve system of claim 1, wherein the flexible material comprises at least one of polytetrafluoroethylene, urethane or silicone.

4. The valve system of claim 1, further comprising at least one gear in the hub, wherein the pinion is coupled to the proximal end of the valve through said at least one gear.

5. The valve system of claim 1, wherein the valve is reinforced with one or more members comprising at least one of stainless steel, nitinol or nylon.

6. The valve system of claim 1, wherein the valve lumen has an inner diameter ranging from 0.01 inches to 2 inches.

7. The valve system of claim 1, wherein the valve lumen has a wall thickness ranging from 0.0002 inches to 0.125 inches.

8. The valve system of claim 1, wherein the valve lumen has a length ranging from 0.01 inches to 2 inches.

9. The valve system of claim 1, further comprising at least one gear in the hub, wherein the rack is coupled to the pinion through said at least one gear.

10. The valve system of claim 1, wherein the valve lumen is configured to be rotated by an angle ranging from 90 degrees to 360 degrees in order to place the lumen in the first state.

11. A valve system for an aspiration catheter, the system comprising:
a valve having a proximal end and a distal end, wherein the distal end of the valve is fixedly coupled to the aspiration catheter, wherein the valve comprises a tube having a flexible lumen that extends between the proximal end and the distal end, and wherein the lumen is in a first constricted state; and
a hub having a body comprising:
a rack;
a pinion engaged with the rack;
a stepped-up gear engaged with the pinion; and
an actuator positioned on the body, wherein the rack is coupled to the actuator and the stepped-up gear is coupled to the proximal end of the valve; and
wherein the actuator, rack, pinion, and stepped-up gear are configured such that applying a force to the actuator displaces the rack along a linear axis which rotates the pinion in a first direction and moves the pinion along the rack, which, in turn, causes the stepped-up gear to rotate in the first direction, which, in turn, causes the proximal end to rotate relative to the distal end, and wherein said relative rotation puts said flexible lumen in a second open state.

12. The valve of claim 11, wherein the first constricted state creates a seal around the aspiration catheter.

13. The valve of claim 11, wherein the second open state allows flow of fluid or passage of devices to the aspiration catheter.

14. The valve system of claim 11, wherein a material of the tube comprises at least one of polytetrafluoroethylene, urethane or silicone.

15. The valve system of claim 14, wherein the tube further comprises at least one of stainless steel members, nitinol members or Nylon members.

16. The valve system of claim 11, wherein the flexible lumen has an inner diameter ranging from 0.01 inches to 2 inches.

17. The valve system of claim 11, wherein the flexible lumen has a wall thickness ranging from 0.0002 inches to 0.125 inch.

18. The valve system of claim 11, wherein the flexible lumen has a length ranging from 0.01 inches to 2 inches.

19. The valve system of claim 11, wherein the actuator is coupled to the rack such that removing a pressure or physical force from the actuator automatically moves the pinion along the rack causing the stepped-up gear to rotate in a second direction opposite to the first direction, wherein the rotation of the stepped-up gear in the second direction causes the proximal end of the valve to rotate relative to the distal end, and wherein said relative rotation puts said flexible lumen back in the first constricted state.

20. The valve system of claim 19, wherein the flexible lumen is configured to be rotated by an angle ranging from 90 degrees to 360 degrees in order to constrict the lumen in the first state.
